(19) United States
(12) Patent Application Publication
(10) Pub. No.: US 2008/0214566 A1
(43) Pub. Date: Sep. 4, 2008

(54) Amorphous ziprasidone mesylate

(76) Inventors: Judith Aronhime, Rehovot (IL); Marioara Mendelovici, Rechovot (IL); Sigalit Levi, Modi'in (IL); Alex Mainfeld, Kfar-Saba (IL); Amir Gold, Herzelia (IL)

Correspondence Address: KENYON & KENYON LLP, ONE BROADWAY, NEW YORK, NY, 10004, US

(21) Appl. No.: 12/151,086

(22) Filed: May 2, 2008

Related U.S. Application Data
(62) Division of application No. 11/354,325, filed on Feb. 13, 2006.
(60) Provisional application No. 60/652,356, filed on Feb. 11, 2005.

(51) Int. Cl. A61K 31/501; A61P 25/18

(52) U.S. Cl. 514/254.02

(57) ABSTRACT

Provided is amorphous form of ziprasidone mesylate and process for its preparation. Also provided is a process for preparing ziprasidone mesylate dihydrate needle crystals.

This application is a divisional of U.S. patent application Ser. No. 11/354,325, filed Feb. 13, 2006, claims the benefit of U.S. Provisional Application No. 60/652,356, filed Feb. 11, 2005, which is herein incorporated by reference.

### FIELD OF THE INVENTION

The invention is directed to amorphous ziprasidone mesylate and processes for its preparation and to a process for preparing ziprasidone mesylate dihydrate needle crystals.

### BACKGROUND OF THE INVENTION

Ziprasidone is an antipsychotic agent and is therefore useful for treating various disorders including schizophrenia, anxiety and migraine pain. Ziprasidone has the following structure:

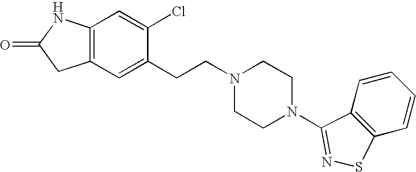

Ziprasidone is marketed under the name GEODON as an oral capsule and as an injectable drug. GEODON capsules contain the monohydrate hydrochloride salt of ziprasidone, and come in 20, 40, 60 and 80 mg dosage forms. GEODON for injection contains a lyophilized form of ziprasidone mesylate trihydrate, and contains 20 mg base equivalent of ziprasidone. The preparation of ziprasidone base is disclosed in U.S. Pat. No. 4,831,031 (example 16). The preparation of ziprasidone base is also disclosed in U.S. Pat. No. 5,312,925.

The present invention relates to the solid state physical properties of ziprasidone mesylate. These properties may be influenced by controlling the conditions under which ziprasidone mesylate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, and differential scanning calorimetry (DSC) and may be used to distinguish some forms from others. A particular form may also give rise to distinct spectroscopic properties that may be detectable by powder X-ray crystallography, solid state ¹³C NMR spectrometry and infrared spectrometry.

U.S. Pat. No. 6,245,765 discloses dihydrate crystalline salts of ziprasidone mesylate and their use as dopamine antagonists. U.S. Pat. No. 6,110,918 discloses that four known ziprasidone mesylate crystalline forms exist. Each crystal form may be characterized by a distinct X-ray powder diffraction pattern and a distinct crystal shape that can be observed by photomicrograph. U.S. Pat. No. 6,110,918 also reports that the ziprasidone mesylate dihydrate lath crystals and dihydrate needle crystals are relatively long and thin in contrast to the prism crystals of ziprasidone mesylate trihydrate. In an aqueous medium at ambient temperature, ziprasidone mesylate trihydrate is reported to be the most thermodynamically stable form of the four crystalline forms of ziprasidone mesylate. U.S. Pat. No. 6,399,777 discloses the preparation of ziprasidone mesylate anhydrous form by adding methanesulfonic acid to a slurry of ziprasidone base in isopropyl alcohol. US 777' also discloses compositions of ziprasidone salt in a cyclodextrin.

The prior art consists of various crystalline forms of ziprasidone mesylate. Because crystalline forms are ordered solids, it often takes more energy to dissolve them as compared to unordered, amorphous forms. As a result, amorphous forms often have greater bioavailability than crystalline forms and may be more suitable for formulation of an active pharmaceutical ingredient when greater bioavailability is desired. There is a need in the art for amorphous forms of ziprasidone mesylate and/or processes for their preparation.

### SUMMARY OF THE INVENTION

In one aspect, the present invention encompasses amorphous ziprasidone mesylate. Preferably, the amorphous ziprasidone mesylate contains less than about 10% of crystalline material, as percentage area XRD. More preferably, the amorphous ziprasidone mesylate contains less than about 5% crystalline materials. Most preferably, the amorphous ziprasidone mesylate contains less than about 1% crystalline materials.

In another aspect, the present invention encompasses amorphous ziprasidone mesylate free of polymer. Preferably, the amorphous ziprasidone mesylate is free of vinyl polymer and/or acrylate polymer. More preferably, the amorphous ziprasidone mesylate is free of cyclodextrin.

In another aspect, the present invention encompasses a process for preparing ziprasidone mesylate dihydrate needle crystals comprising drying wet ziprasidone mesylate dihydrate lath crystals. Preferably, the ziprasidone mesylate dihydrate lath crystals are dried at a temperature of from about 40° C. to about 50° C., more preferably at a temperature of about 45° C. Preferably, the ziprasidone mesylate dihydrate lath crystals are dried at a pressure below about 100 mg Hg. Preferably, the ziprasidone mesylate dihydrate lath crystals are dried for more than about 24 hours. More preferably, the ziprasidone mesylate dihydrate lath crystals are dried for about 2 days.

In another aspect, the present invention provides a process for preparing amorphous ziprasidone mesylate comprising heating ziprasidone mesylate dihydrate needle crystal for about ten minutes to about 1 hour, at a temperature of above about 100° C. More preferably, ziprasidone mesylate dihydrate crystals are heated at a temperature above about 140° C. Most preferably, ziprasidone dihydrate crystals are heated at a temperature of about 140° C. to about 160° C.

In another aspect, the present invention encompasses a pharmaceutical formulation comprising amorphous ziprasidone mesylate and at least one pharmaceutically acceptable excipient.

In another aspect, the present invention encompasses a method of treating a patient suffering from schizophrenia comprising administering to the patient a therapeutically effective amount of the pharmaceutical formulation.

### BRIEF DESCRIPTION OF THE DRAWINGS

FIG. 1 illustrates the x-ray diffraction pattern of amorphous ziprasidone mesylate.

FIG. 2 illustrates the DSC thermogram of amorphous ziprasidone mesylate.

### DETAILED DESCRIPTION OF THE INVENTION

In one aspect, the present invention provides amorphous ziprasidone mesylate. In contrast to crystalline forms, amorphous solids consist of disordered arrangements of molecules and do not possess a distinguishable crystal lattice. Amorphous forms are desirable because the bioavailability of a compound may be increased relative to its crystalline forms, as generally amorphous forms are more soluble.

Preferably, the amorphous ziprasidone mesylate is free of polymer. As used herein “free of polymer” refers to a free drug, in which the solid particles of compound are not intimately embedded in polymeric co-precipitate. More preferably, the amorphous ziprasidone mesylate is free of vinyl polymers and acrylate polymers, and, most preferably, free of cyclodextrins.

Preferably, the amorphous ziprasidone mesylate is a solid state where the solid contains less than about 10% crystalline materials. More preferably, amorphous forms contain less than about 5% crystalline materials. Most preferably, amorphous forms contain less than about 1% crystalline materials. Presence of amorphous forms may be assessed by lack of peaks in a powder XRD pattern or lack of a melting peak in a DSC thermogram. The area under the peaks in an XRD pattern may be added to obtain total amount of crystalline material. With DSC, presence of endotherms may point to melting of crystalline material.

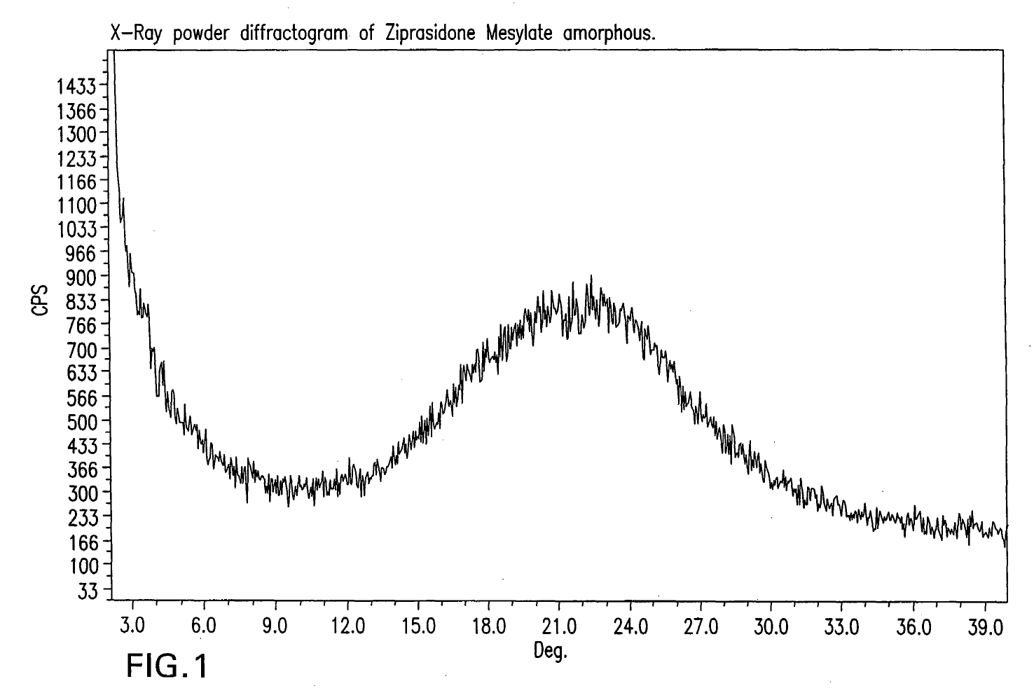

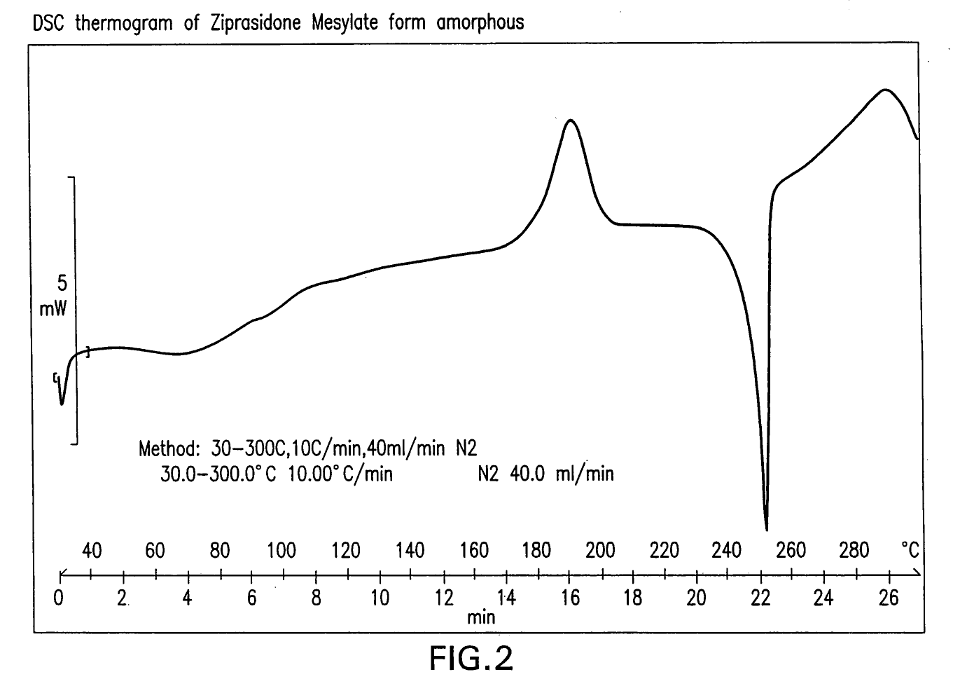

As one skilled in the art will appreciate, amorphous form lacks a crystal structure and thus lacks X-Ray powder diffraction peaks. As depicted in FIG. 1, reflection peaks are substantially absent (halo-like pattern) in the XRD of amorphous ziprasidone mesylate. The DSC thermogram of amorphous ziprasidone mesylate is illustrated in FIG. 2. FIG. 2 shows an exothermic peak at about 190° C., which is due to the crystallization of the amorphous form. A sharp endothermic peak of the crystallized material is observed at about 250° C. This is the melting peak of the crystallized material.

In another aspect, the present invention provides a process for preparing ziprasidone mesylate dihydrate needle crystals comprising drying wet ziprasidone mesylate dihydrate lath crystals.

The ziprasidone mesylate dihydrate lath crystals may be prepared by the process disclosed in U.S. Pat. No. 6,245,765, herein incorporated by reference.

Preferably, the ziprasidone mesylate dihydrate lath crystals are dried at a temperature of from about 40° C. to about 50° C. at a pressure below about 100 mm Hg in a vacuum oven for more than about 24 hours. More preferably, the ziprasidone mesylate dihydrate lath crystals are dried at a temperature of about 45° C. for about 2 days.

In another aspect, the present invention provides a process for preparing amorphous ziprasidone mesylate by heating ziprasidone mesylate dihydrate needle crystal.

Preferably, the ziprasidone mesylate is heated to a temperature of above about 100° C. More preferably, the ziprasidone mesylate is heated to a temperature of above about 140° C. Most preferably, ziprasidone mesylate is heated to a temperature of from about 140° C. to about 160° C.

One skilled in the art will appreciate that the heating time to obtain amorphous ziprasidone mesylate will vary depending on, among other things, the quantity of the reaction and the temperature of heating. One skilled in the art can determine the time to obtain amorphous form by, for example, monitoring by XRD or DSC during heating to determine whether a transformation has occurred. Preferably, the time sufficient to obtain amorphous ziprasidone mesylate is from about ten minutes to about one hour.

The present invention also provides pharmaceutical compositions containing amorphous ziprasidone mesylate, optionally in mixture with other form(s) of ziprasidone. In addition to the active ingredient(s), the pharmaceutical formulations of the present invention contain one or more excipients. Excipients are added to the formulation for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, ziprasidone and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

The dosage of GEODON may be used as a guidance and with routine experimentation one can determine the appropriate dosage of the present invention. The oral dosage form of the present invention is preferably in the form of an oral capsule having a dosage of about 10 mg to about 160 mg, more preferably of about 20 mg to about 80 mg, and most preferably capsules of 20, 40, 60 and 80 mg. An injectable dosage preferably contains a dosage equivalent to about 5 to about 80 mg of ziprasidone base, and more preferably contains a dosage equivalent to about 10 to about 40 mg of ziprasidone base. Most preferably, an injectable dosage contains a dosage equivalent to about 20 to about 30 mg of ziprasidone base.

The present invention also provides a method of treating a patient suffering from schizophrenia comprising administering to the patient a pharmaceutical composition of amorphous ziprasidone mesylate in a therapeutically effective amount.

Having described the invention with reference to certain preferred embodiments, other embodiments will become apparent to one skilled in the art from consideration of the specification. The invention is further defined by reference to the following examples describing in detail the preparation of ziprasidone mesylate amorphous form. It will be apparent to those skilled in the art that many modifications, both to materials and methods, may be practiced without departing from the scope of the invention.

### EXAMPLES

### Experimental

X-Ray powder diffraction data were obtained using a SCINTAG powder X-Ray diffractometer model X'TRA equipped with a solid state detector. Copper radiation of 1.5418 Å was used. A round aluminum sample holder with zero background was used. The scanning parameters included: range: 2-40 degrees two-theta; scan mode: continuous scan; step size: 0.05 deg and a rate of 5 deg/min. All peak positions are within ±0.2 degrees two theta.

DSC analysis was performed using a Mettler 821 Stare. The weight of the samples is about 3-6 mg; the samples were scanned at a rate of 10° C./min from 30° C. to at least 300° C. The oven is constantly purged with nitrogen gas at a flow rate of 40 ml/min. Standard 40 μl aluminum crucibles covered by lids with 3 holes were used.

### Example 1

### Preparation of Ziprasidone Mesylate Dihydrate Needle Crystals from Ziprasidone Mesylate Dihydrate Lath Crystals

Wet ziprasidone mesylate dihydrate lath crystals were prepared according to U.S. Pat. No. 6,245,765. The wet needle crystals were dried in a vacuum oven at about 45° C. for about 2 days to obtain ziprasidone mesylate dihydrate needle crystals.

### Example 2

### Preparation of Ziprasidone Mesylate Amorphous Form

Ziprasidone mesylate dihydrate needle crystals were heated at about 140° C. for about 30 minutes to obtain ziprasidone mesylate amorphous form.

### Example 3

### Preparation of Ziprasidone Mesylate Amorphous Form

Ziprasidone mesylate dihydrate needle crystals were heated at about 160° C. for about 30 minutes to obtain ziprasidone mesylate amorphous form.

What is claimed is:

1. A method of treating a patient suffering from schizophrenia comprising
administering to the patient the pharmaceutical composition of Amorphous ziprasidone mesylate and at least one pharmaceutically acceptable excipient in a therapeutically effective amount.

2. The method of claim 1, wherein the amorphous ziprasidone mesylate contains less than about 10% of crystalline material as percentage area XRD.

3. The method of claim 1, wherein the amorphous ziprasidone mesylate contains less than about 5% of crystalline material as percentage area XRD.

4. The method of claim 1, wherein the amorphous ziprasidone mesylate contains less than about 1% of crystalline material as percentage area XRD.

5. The method of claim 1, wherein the amorphous ziprasidone mesylate is free of polymer.

6. The method of claim 5, wherein the polymer is selected from the group consisting of vinyl polymer and acrylate polymer.

7. The method of claim 6, wherein the polymer is cyclodextrin.

8. The method of claim 1, wherein the amorphous form is prepared by a process comprising heating ziprasidone mesylate dihydrate needle crystals.

9. The method of claim 8, wherein the ziprasidone mesylate is heated to a temperature of above about 100° C.

10. The method of claim 9, wherein the ziprasidone mesylate is heated to a temperature of above about 140° C.

11. The method of claim 10, wherein the ziprasidone mesylate is heated to a temperature of from about 140° C. to about 160° C.

12. The method of claim 8, wherein the ziprasidone mesylate is heated for from about ten minutes to about 1 hour.
